(19) United States
(12) Patent Application Publication
(10) Pub. No.: US 2021/0090698 A1
(43) Pub. Date: Mar. 25, 2021

(54) METHOD AND SYSTEM OF GENERATING PATIENT MEDICAL RECORD DATASET

(71) Applicant: Ventech Solutions, Inc., Columbus, OH (US)

(72) Inventors: Ravi Kiran Pasupuleti, Triplicane Chennai (IN); Ravi Kunduru, Columbus, OH (US)

(21) Appl. No.: 16/577,005

(22) Filed: Sep. 20, 2019

(51) Int. Cl. G16H 15/00; G06K 9/46; G16H 40/67

(52) U.S. Cl. CPC G16H 15/00; G06K 2209/01; G16H 40/67; G06K 9/46

(57) ABSTRACT

A method and system of generating a patient medical record dataset. The method comprises receiving, at a server computing device, image data acquired at a mobile computing device in association with medical monitoring of a patient state, deriving patient diagnostic data based on interpreting the image data, and formatting the patient diagnostic data in accordance with a longitudinal dataset associated with a patient medical record.

### TECHNICAL FIELD

The disclosure herein relates to the field of generating patient medical records.

### BACKGROUND

Mobile device are being carried and deployed with increasing levels of functionality by individuals. Patient medical data is generally acquired at designated patient medical facilities under relatively structured circumstances. Under both structured and certain spontaneous circumstances, mobile device acquired data may be deployed to augment, and even more comprehensively to derive, data being generated for patient medical records.

### BRIEF DESCRIPTION OF THE DRAWINGS

FIG. 1 illustrates, in an example embodiment, a system for generating a patient medical record dataset;

FIG. 2 illustrates an example architecture of a computing and communication mobile device for acquisition of data associated with monitoring a medical patient;

FIG. 3 illustrates an example architecture of a server computing device for generating a patient medical record dataset; and

FIG. 4 illustrates, in an example embodiment, a method of operation in generating a patient medical record dataset.

### DETAILED DESCRIPTION

Embodiments herein recognize that, in order for a patient's medical record to be comprehensive, medical events that occur even in non-medically-related surroundings or stations should be captured. Such medical events, although occurring spontaneously rather than in a more structured medical facility, are important to creating and generating a more comprehensive medical record for a given patient and can result in most effective diagnoses and treatments. Among other technical effects and advantages, embodiments herein provide solutions which are directed to capturing patient-related medical data in spontaneous as well as relatively structured surroundings and circumstances. Embodiments herein also provide for generating the medical record configured in a longitudinal dataset where medical events are directly temporally linked, advantageously resulting in more efficient medical record query searches.

Provided is a method of generating a patient medical record dataset. The method comprises receiving, at a server computing device, image data acquired at a mobile computing device in association with medical monitoring of a patient state, deriving patient diagnostic data based on interpreting the image data, and formatting the patient diagnostic data in accordance with a longitudinal dataset associated with a patient medical record.

Also provided is a server computing system for generating a patient medical record dataset. The system includes one or more processors and a memory storing executable instructions. The instructions are executable to receive, at a server computing device, image data acquired at a mobile computing device in association with medical monitoring of a patient state, derive patient diagnostic data based on interpreting the image data, and format the patient diagnostic data in accordance with a longitudinal dataset associated with a patient medical record.

Further provided is a non-transitory memory medium storing instructions executable in one or more processor devices. The instructions are executable to receive, at a server computing device, image data acquired at a mobile computing device in association with medical monitoring of a patient state, derive patient diagnostic data based on interpreting the image data, and format the patient diagnostic data in accordance with a longitudinal dataset associated with a patient medical record.

One or more embodiments described herein provide that methods, techniques, and actions performed by a computing device are performed programmatically, or as a computer-implemented method. Programmatically, as used herein, means through the use of code or computer-executable instructions. These instructions can be stored in one or more memory resources of the computing device. A programmatically performed step may or may not be automatic.

One or more embodiments described herein can be implemented using programmatic modules, engines, or components. A programmatic module, engine, or component can include a program, a sub-routine, a portion of a program, or a software component or a hardware component capable of performing one or more stated tasks or functions. As used herein, a module or component can exist on a hardware component independently of other modules or components. Alternatively, a module or component can be a shared element or process of other modules, programs or machines.

Some embodiments described herein can generally require the use of computing devices, including processor and memory resources. For example, one or more embodiments described herein may be implemented, in whole or in part, on computing devices such as servers, desktop computers, mobile devices including cellular or smartphones, laptop computers, wearable devices, and tablet devices. Memory, processing, and network resources may all be used in connection with the establishment, use, or performance of any embodiment described herein, including with the performance of any method or with the implementation of any system.

Furthermore, one or more embodiments described herein may be implemented through the use of instructions that are executable by one or more processors. These instructions may be carried on a computer-readable medium. Machines shown or described with figures below provide examples of processing resources and computer-readable mediums on which instructions for implementing embodiments of the invention can be carried and/or executed. In particular, the numerous machines shown with embodiments of the invention include processor(s) and various forms of memory for holding data and instructions. Examples of computer-readable mediums include permanent memory storage devices, such as hard drives on personal computers or servers. Other examples of computer storage mediums include portable memory storage units, flash memory (such as carried on smartphones, multifunctional devices or tablets), and magnetic memory. Computers, terminals, network enabled devices (e.g., mobile devices, such as cell phones) are all examples of machines and devices that utilize processors, memory, and instructions stored on computer-readable mediums. Additionally, embodiments may be implemented in the form of computer-programs, or a computer usable carrier medium capable of carrying such a program.

### System Description

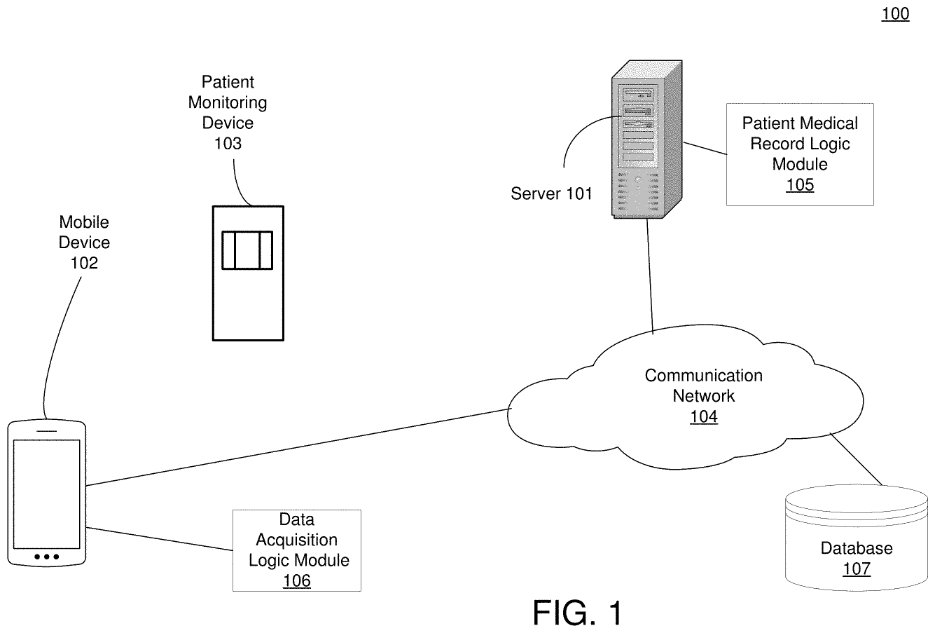

FIG. 1 illustrates, in an example embodiment, a system for generating and deploying a patient medical record dataset. Server 101 includes patient medical record logic module 105 and is communicatively connected via communication network 104 to a computing and communication mobile device 102, and to external database 107. Mobile device 102 includes data acquisition logic module 106, which in one embodiment, may include user navigation and positioning capability based on various sensor devices and associated functionality incorporated therein.

Mobile device 102, in embodiments, incorporates a camera, wireless signal strength (WiFi and BLUETOOTH), inertial (gyroscope and accelerometer), barometric and magnetic sensors in conjunction with associated functionality, including but not limited to mobile device localization functionality. Patient monitoring device 103, in some embodiments including such as a blood pressure measurement device or a cardiovascular parameter measurement machine, provides medical parameter measured values associated with monitoring a medial state of a patient. The camera functionality of mobile device 102 may be used to take a picture of a medical instrument or device reading rendered at a display interface of patient monitoring device 103.

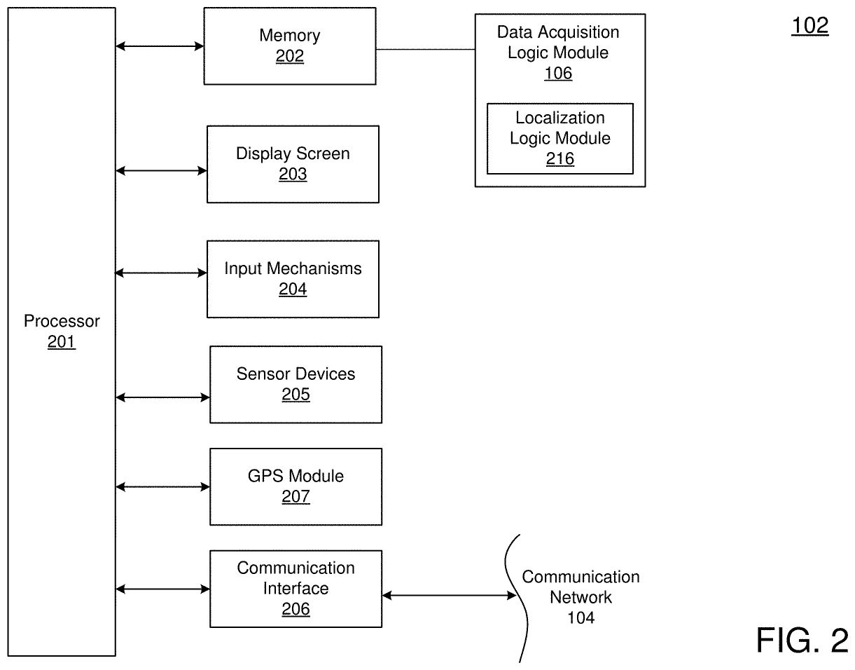

FIG. 2 illustrates an example architecture of a computing and communication mobile device 102, representative of a plurality of mobile device 102 for acquisition of fingerprint data in conjunction with GPS data for particular positions or locations within the indoor area. In embodiments, mobile device 102 may correspond to, for example, a cellular communication device (e.g., smartphone, tablet, etc.) that is capable of telephony, messaging, and data computing functionality and services. In variations, mobile device 102 can correspond to, for example, a tablet computing device or a wearable computing device. Mobile device 102 may include processor 201, memory 202, display interface or screen 203, input mechanisms 204 such as a keyboard or software-implemented touchscreen input functionality, barcode, QR code or other symbol- or code-scanner input functionality. Mobile device 102 may include global positioning system (GPS) functionality. Mobile device 102 may include sensor functionality by way of sensor devices 205. Sensor devices 205 may include any of inertial sensors (accelerometer, gyroscope), magnetometer or other magnetic field sensing functionality, and barometric or other environmental pressure sensing functionality. Mobile device 102 may also include capability for detecting and communicatively accessing wireless communication signals, including but not limited to any of BLUETOOTH, Wi-Fi, RFID, and GPS signals via GPS module 207. Mobile device 102 further includes capability for detecting and measuring a received signal strength of the wireless communication signals. In particular, mobile device 102 may include communication interface 206 for communicatively coupling to communication network 104, such as by sending and receiving cellular data over data channels and voice channels.

Data acquisition logic module 106 includes instructions stored in memory 202 of mobile device 102. In embodiments, data acquisition logic module 106 includes stored localization logic module 216 comprising processor-executable instructions stored in memory 202 of mobile device 102 for acquiring fingerprint data unique to specific locations or positions within a building. The indoor infrastructure may be a hospital, a residential building, a medical clinic, a shopping mall, an airport, a warehouse, a university, or any at least partially enclosed building.

Acquisition of fingerprint data associated with a unique position or location with an indoor building, based on the sensor functionality incorporated into mobile device 102 may be automatically triggered, or concurrently performed, at mobile device 102 upon an event occurrence. The event occurrence in an embodiment consists of a user of mobile device 102 taking a picture of medical monitoring instrument during monitoring of a patient state in which a state or condition of the patient is measured and rendered. The measured state, for instance a blood pressure reading or a cardiopulmonary related reading, may be rendered at a display interface of patient monitoring device 103. An image captured and created upon taking the picture using a camera of the mobile device 102 may be associated with various contextual and patient specific data, then transmitted or uploaded to memory 302 of server device 101. It is contemplated that the image capture relates to patient monitoring device 103 being, in various embodiments, any one or more of a blood glucose meter, a pacemaker, an insulin pump, a pulse oximeter, an electrocardiograph, a defibrillator, an electroencephalograph, a blood alcohol monitor, an alcohol breathalyzer, an alcohol vehicle ignition interlock, a respiration monitor, a skin galvanometer, a thermometer, a medical patient geolocation device, a patient weighing scale, an intravenous flow regulator, a patient height measurement device, a biochip device, a biological agent monitoring device; a hazardous chemical agent monitoring device; a radiation sensor, and a medical or health event monitor.

The term mobile device localization as used herein refers to determining a position of the mobile device, specifically in terms of global or local (X,Y,Z) coordinates, using functionality of the various sensors incorporated. The localization of mobile device 102 may be based at least in part using sensor devices 205 of the mobile device 102, including but not limited to an accelerometer, a gyroscope, a magnetometer, a barometer, and a wireless signal strength sensor. A data fusion of the various sensor data in conjunction with a kalman filtering process, for example, may include any one of inertial and orientation data, magnetic field data including strength and direction, received wireless signal strength data, barometric pressure data, and also GPS location data at a given position within an indoor building equipped with wireless signaling infrastructure.

In some embodiments, localization may also include determining a floor within the building, and thus involve determining not only horizontal planar (X,Y) coordinates, but also include a vertical, or z, coordinate of the mobile device, the latter embodying a floor number within a multi-floor building or multi-level building, for example, in accordance with barometric fingerprint data acquired at mobile device 102. In other embodiments, the (X,Y,Z) coordinates may be expressed either in a local reference frame specific to the mobile device, or in accordance with a global coordinate reference frame.

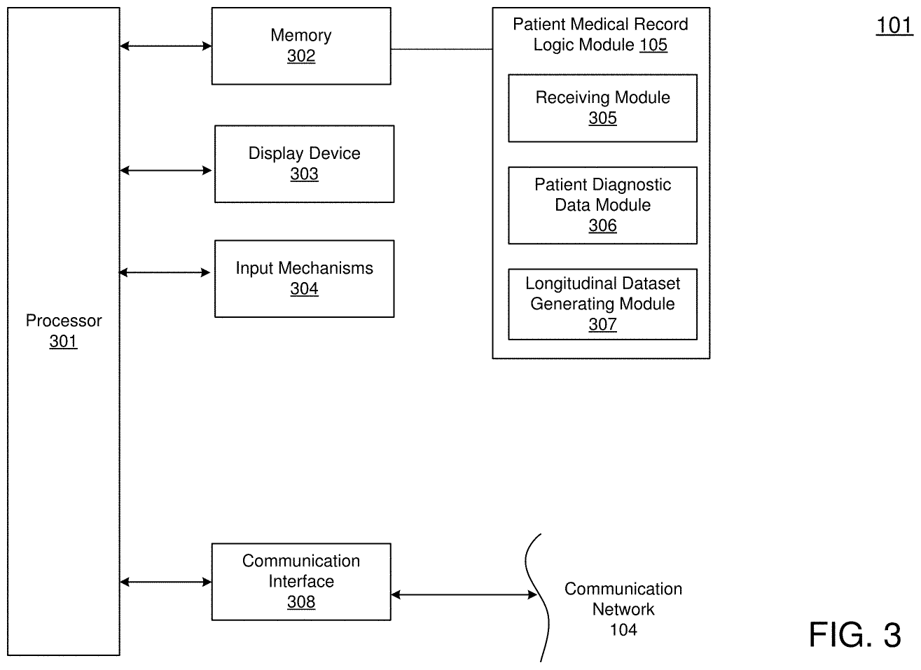

FIG. 3 illustrates an example architecture of a server computing device for generating a patient medical record dataset. Server 101, in an embodiment architecture, may be implemented on one or more server devices, and includes processor 301, memory 302 which may include a read-only memory (ROM) as well as a random access memory (RAM) or other dynamic storage device, display device 303, input mechanisms 304 and communication interface 308 for communicative coupling to communication network 104. Processor 301 is configured with software or other logic, such as comprised of patient medical record logic module 105, to perform one or more processes, steps and other functions described with implementations herein, including as described in conjunction with FIGS. 1 through 4 herein. Processor 301 may process information and instructions stored in memory 302, such as provided by a random access memory (RAM) or other dynamic storage device, for storing information and instructions which are executable by processor 301. Memory 302 also may be used for storing temporary variables or other intermediate information during execution of instructions to be executed by processor 301. Memory 302 may also include the ROM or other static storage device for storing static information and instructions for processor 301; a storage device, such as a magnetic disk or optical disk, may be provided for storing information and instructions. Communication interface 308 enables server 101 to communicate with one or more communication networks 104 (e.g., cellular network) through use of the network link (wireless or wired). Using the network link, server 101 can communicate with mobile device 102.

Patient medical record logic module 105 of server 101 includes instructions stored in RAM of memory 302, and includes receiving module 305, patient diagnostic data module 306, and longitudinal dataset generating module 307.

Processor 301 uses executable instructions stored in receiving module 305 to receive, at a server computing device, image data acquired at a mobile computing device in association with medical monitoring of a patient state.

Associated with a given image data, fingerprint data associated with a given position may be acquired at mobile device 102, for instance at time of capturing an image, such as a photograph, of a medical parameter measurement displayed at medical monitoring device 103. The fingerprint data may include at least two of wireless signal data, inertial data, magnetic data, barometric data and optical data that are time-stamped and time-correlated for respective positions in unique positions within an indoor building. The fingerprint data may be used to localize mobile device 102 in association at time of image capture using the camera of mobile device 102.

In embodiments, the fingerprint data, as acquired from the mobile device, further includes respective time-stamps, whereby the orientation and other inertial sensor data, the magnetic field strength and direction, the received wireless signal strength, the barometric pressure, and the position data can be time-correlated in accordance with the time-stamps with respect to any given coordinate position.

Processor 301 uses executable instructions stored in patient diagnostic data module 306 to derive patient diagnostic data based on interpreting the image data.

In embodiments, the patient diagnostic or measurement data may be interpreted from the image data based at least in part upon an optical character recognition (OCR) engine, providing a digital measurement record thereof.

In variations, interpreting the image data using the OCR engine provides the patient state measurement and also identifies one or more of a model identification number including a serial number and a model type of the measurement instrument used for the medical monitoring. Image characteristics, such as represented by a photograph, of the respective patient being monitored using patient monitoring device 103 may be optionally provided within, or in conjunction with, the image data. A facial recognition engine may be used to correlate the image data, as well as medical records derived therefrom, with an identified patient.

In some embodiments, the image data acquired at the mobile device may be provided with a set of coordinates of a location or position associated therewith. The position or location provided may be based on fingerprint data including at least two of wireless signal data, inertial data, magnetic data, barometric data and optical data that are time-stamped and time-correlated for respective positions. The fingerprint data may applied as input to a data fusion process that provides an output coordinate location associated with acquisition of the image data. A set of (X, Y, Z) coordinates of the location may include a height and a depth coordinate, representing a distance above or below a sea level or a local ground level in accordance with the barometric fingerprint data of mobile device 102.

In other embodiments, server 101 accesses a floor layout of an indoor infrastructure, for example in conjunction with database 107, that includes the location of image capture. The indoor infrastructure may be such as a hospital, a medical clinic, a shopping mall, an airport, a residential building, a campus building or any at least partially enclosed building. Based at on the set of coordinates, server 101 may determine, in accordance with either the height or the depth coordinate and the floor layout, a floor number designation, and optionally even a functional departmental designation, such as, but not limited to, a medical operation theatre, within the indoor infrastructure that is associated with the image data.

The position or location associated with the mobile device as used herein refers to a coordinate location and may be expressed in local or global (X, Y) coordinate terms. In some embodiments, the coordinates may further include a Z coordinate representing a height, or a negative Z coordinate representing a depth, in accordance with barometric fingerprint data acquired at mobile device 102. The vertical, or Z-coordinate, for example may be associated with a given floor relative to a ground floor within a multi-floor building, the coordinates including a set of (X, Y, Z) coordinate terms as determined from localizing the mobile device at time of acquiring or transmitting the image data.

Processor 301 uses executable instructions stored in longitudinal dataset generating module 307 to format the patient diagnostic data in accordance with a longitudinal dataset associated with a patient medical record.

In embodiments, the longitudinal dataset establishes a temporal relationship among longitudinally structured data associated with cumulative medical monitoring data of a set of patient states comprising the patient medical record. The temporal relationship comprises classification of search parameters with a discrete event from a set of medical events associated with the set of patient states.

Further embodiments include structuring the longitudinal dataset in accordance with a graph data structure that includes a tables graph, where each table of the tables graph is represented as a table node. In variations, wherein the table node is configured as either sparsely-connected connected, fully-connected, or including a variable number of connections to other table nodes.

In such embodiments, based on the search parameters, a query engine can efficiently traverse the tables graph to determine how to incorporate data relevant to the temporally graphed specific medical states, events or conditions for a query requestor.

### Methodology

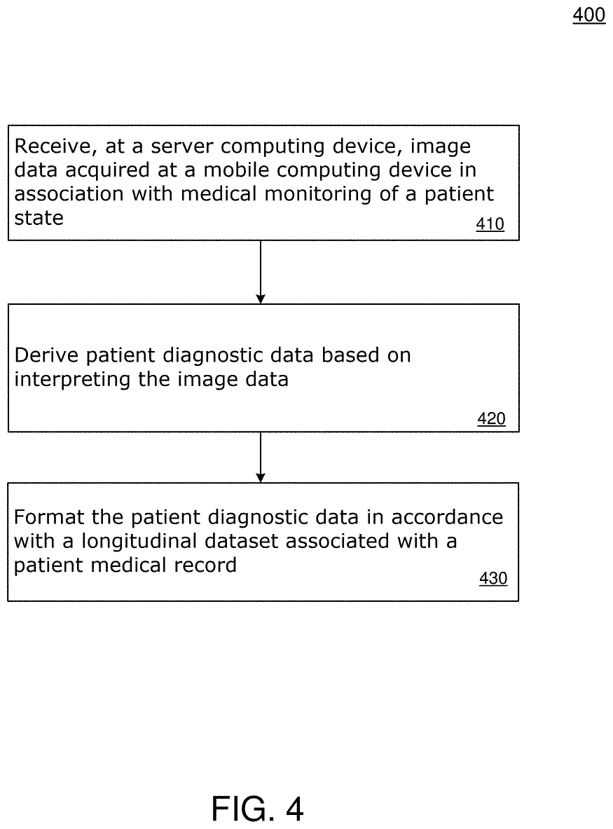

FIG. 4 illustrates, in an example embodiment, a method of operation 400 in generating a patient medical record dataset. In describing examples of FIG. 4, reference is made to the examples of FIGS. 1-3 for purposes of illustrating suitable components or elements for performing a step or sub-step being described.

Examples of method steps described herein are related to the use of server 101 for implementing the techniques described herein. According to one embodiment, the techniques are performed by patient medical record logic module 105 of server 101 in response to the processor 301 executing one or more sequences of software logic instructions that constitute patient medical record logic module 105. In embodiments, patient medical record logic module 105 may include the one or more sequences of instructions within sub-modules including receiving module 305, patient diagnostic data module 306 and longitudinal dataset generating module 307. Such instructions may be read into memory 302 from machine-readable medium, such as memory storage devices. Execution of the sequences of instructions contained in receiving module 305, patient diagnostic data module 306 and longitudinal dataset generating module 307 of patient medical record logic module 105 in memory 302 causes processor 301 to perform the process steps described herein. In alternative implementations, at least some hard-wired circuitry may be used in place of, or in combination with, the software logic instructions to implement examples described herein. Thus, the examples described herein are not limited to any particular combination of hardware circuitry and software instructions.

At step 410, processor 301 executes the instructions of receiving module 305, to receive, at memory 302 of server computing device 101, image data acquired at mobile computing device 102 in association with medical monitoring of a patient state

Associated with a given image data, fingerprint data associated with a given position may be acquired at mobile device 102, for instance at time of capturing an image, such as a photograph, of a medical parameter measurement displayed at medical monitoring device 103. The fingerprint data may include at least two of wireless signal data, inertial data, magnetic data, barometric data and optical data that are time-stamped and time-correlated for respective positions in unique positions within an indoor building. The fingerprint data may be used to localize mobile device 102 in association at time of image capture using the camera of mobile device 102.

In embodiments, the fingerprint data, as acquired from the mobile device, further includes respective time-stamps, whereby the orientation and other inertial sensor data, the magnetic field strength and direction, the received wireless signal strength, the barometric pressure, and the position data can be time-correlated in accordance with the time-stamps with respect to any given coordinate position.

At step 420, processor 301 executes the instructions of patient diagnostic data module 306 to derive patient diagnostic data based on interpreting the image data.

In embodiments, the patient diagnostic or measurement data may be interpreted from the image data based at least in part upon an optical character recognition (OCR) engine, providing a digital measurement record thereof.

In variations, interpreting the image data using the OCR engine provides the patient state measurement and also identifies a model identification number and a model type of the measurement instrument used in the medical monitoring. Image characteristics, such as represented by a photograph, of the respective patient being monitored using patient monitoring device 103 may be optionally provided within, or in conjunction with, the image data. A facial recognition engine may be used to correlate the image data, as well as medical records derived therefrom, with an identified patient.

In some embodiments, the image data acquired at the mobile device may be provided with a set of coordinates of a location or position associated therewith. The position or location provided may be based on fingerprint data including at least two of wireless signal data, inertial data, magnetic data, barometric data and optical data that are time-stamped and time-correlated for respective positions. The fingerprint data may applied as input to a data fusion process that provides an output coordinate location associated with acquisition of the image data. A set of (X, Y, Z) coordinates of the location may include a height and a depth coordinate, representing a distance above or below a sea level or a local ground level in accordance with the barometric fingerprint data of mobile device 102.

In other embodiments, server 101 accesses a floor layout of an indoor infrastructure, for example in conjunction with database 107, that includes the location of image capture. The indoor infrastructure may be such as a hospital, a medical clinic, a shopping mall, an airport, a residential building, a campus building or any at least partially enclosed building. Based at on the set of coordinates, server 101 may determine, in accordance with either the height or the depth coordinate and the floor layout, a floor number designation, and optionally even a functional departmental designation, such as, but not limited to, a medical operation theatre, within the indoor infrastructure that is associated with the image data.

At step 430, processor 301 executes the instructions of longitudinal dataset generating module 307 to format the patient diagnostic data in accordance with a longitudinal dataset associated with a patient medical record.

In embodiments, the longitudinal dataset establishes a temporal relationship among longitudinally structured data associated with cumulative medical monitoring data of a set of patient states comprising the patient medical record. The temporal relationship comprises classification of search parameters with a discrete event from a set of medical events associated with the set of patient states.

Further embodiments include structuring the longitudinal dataset in accordance with a graph data structure that includes a tables graph, where each table of the tables graph is represented as a table node. In variations, wherein the table node is configured as either sparsely-connected, fully-connected, or including a variable number of connections to other table nodes.

In such embodiments, based on the search parameters, a query engine can efficiently traverse the tables graph to determine how to incorporate data relevant to the temporally graphed specific medical states, events or conditions for a query requestor.

It is contemplated for embodiments described herein to extend to individual elements and concepts described herein, independently of other concepts, ideas or system, as well as for embodiments to include combinations of elements recited anywhere in this application. Although embodiments are described in detail herein with reference to the accompanying drawings, it is to be understood that the invention is not limited to those precise embodiments. As such, many modifications and variations will be apparent to practitioners skilled in this art. Accordingly, it is intended that the scope of the invention be defined by the following claims and their equivalents. Furthermore, it is contemplated that a particular feature described either individually or as part of an embodiment can be combined with other individually described features, or parts of other embodiments, even if the other features and embodiments make no mention of the particular feature. Thus, the absence of describing combinations should not preclude the inventor from claiming rights to such combinations.

What is claimed is:

1. A method comprising:
receiving, at a server computing device, image data acquired at a mobile computing device in association with medical monitoring of a patient state;
deriving patient diagnostic data based on interpreting the image data; and
formatting the patient diagnostic data in accordance with a longitudinal dataset associated with a patient medical record.

2. The method of claim 1 further comprising interpreting the image data based at least in part upon an optical character recognition operation.

3. The method of claim 2 wherein interpreting the image data provides at least a patient state measurement and identifies a measurement instrument associated with the medical monitoring.

4. The method of claim 1 further comprising receiving, in association with the image data acquired at the mobile device, a set of coordinates of a location associated therewith.

5. The method of claim 2 wherein the set of coordinates of the location includes one of a height and a depth coordinate.

6. The method of claim 5 further comprising:
accessing a floor layout of an indoor infrastructure based at least in part on the set of coordinates; and
determining, based at least in part upon the one of the height and the depth coordinate and the floor layout, at least one of a floor designation and a functional departmental designation associated with the indoor infrastructure.

7. The method of claim 6 wherein the indoor infrastructure comprises one of a hospital, a medical clinic, a shopping mall, an airport, a residential building, a campus building and an at least partially enclosed building.

8. The method of claim 1 wherein the longitudinal dataset establishes a temporal relationship among longitudinally structured data associated with cumulative medical monitoring data of a set of patient states comprising the patient medical record.

9. The method of claim 8 wherein the temporal relationship comprises classification of search parameters with a discrete event from a set of events associated with the set of patient states.

10. The method of claim 9 further comprising structuring the longitudinal dataset in accordance with a graph data structure that includes a tables graph, each table of the tables graph being represented as a table node, wherein the table node is one of sparsely-connected, fully-connected, and includes a variable number of connections to other table nodes.

11. A server computing system comprising:
a processor;
a memory storing a set of instructions, the instructions executable in the processor to:
receive, at a server computing device, image data acquired at a mobile computing device in association with medical monitoring of a patient state;
derive patient diagnostic data based on interpreting the image data; and
format the patient diagnostic data in accordance with a longitudinal dataset associated with a patient medical record.

12. The server computing system of claim 11 further comprising instructions executable to interpret the image data based at least in part upon an optical character recognition operation.

13. The server computing system of claim 12 wherein interpreting the image data provides at least a patient state measurement and identifies a measurement instrument associated with the medical monitoring.

14. The server computing system of claim 11 further comprising instructions executable to receive, in association with the image data acquired at the mobile device, a set of coordinates of a location associated therewith.

15. The server computing system of claim 12 wherein the set of coordinates of the location includes one of a height and a depth coordinate.

16. The server computing system of claim 15 further comprising instructions executable to:
access a floor layout of an indoor infrastructure based at least in part on the set of coordinates; and
determine, based at least in part upon the one of the height and the depth coordinate and the floor layout, at least one of a floor designation and a functional departmental designation associated with the indoor infrastructure.

17. The server computing system of claim 16 wherein the indoor infrastructure comprises one of a hospital, a medical clinic, a shopping mall, an airport, a residential building, a campus building and an at least partially enclosed building.

18. The server computing system of claim 11 wherein the longitudinal dataset establishes a temporal relationship among longitudinally structured data associated with cumulative medical monitoring data of a set of patient states comprising the patient medical record.

19. The server computing system of claim 18 wherein the temporal relationship comprises classification of search parameters with a discrete event from a set of events associated with the set of patient states.

20. The server computing system of claim 19 further comprising instructions executable to structure the longitudinal dataset in accordance with a graph data structure that includes a tables graph, each table of the tables graph being represented as a table node, wherein the table node is one of sparsely-connected, fully-connected, and includes a variable number of connections to other table nodes.
